(19) United States
(12) Patent Application Publication
(10) Pub. No.: US 2024/0062671 A1
(43) Pub. Date: Feb. 22, 2024

(54) SIMULATOR AND SIMULATION SYSTEM FOR BRAIN TRAINING BASED ON BEHAVIOR MODELING

(71) Applicant: DAEGU GYEONGBUK INSTITUTE OF SCIENCE AND TECHNOLOGY, Daegu (KR)

(72) Inventors: Jinung AN, Daegu (KR); Sanghyeon JIN, Daegu (KR); Seunghyun LEE, Daegu (KR)

(73) Assignee: DAEGU GYEONGBUK INSTITUTE OF SCIENCE AND TECHNOLOGY, Daegu (KR)

(21) Appl. No.: 18/446,885

(22) Filed: Aug. 9, 2023

Related U.S. Application Data
(63) Continuation of application No. 16/500,955, filed on Dec. 5, 2019.

(30) Foreign Application Priority Data
Apr. 11, 2017 (KR) 10-2017-0046691

(51) Int. Cl. G09B 9/00; A61B 5/00; G09B 19/00; A61B 5/375

(52) U.S. Cl. CPC G09B 9/00; A61B 5/4866; A61B 5/7264; G09B 19/00; A61B 5/375

(57) ABSTRACT

Provided are a brain training simulator and a brain training simulation system. The brain training simulator includes at least one memory, and at least one processor configured to acquire a brain signal of a user acting in a first action state based on a non-invasive brain activation measurement method, determine whether an intention of the user is recognized, by selecting preset intention data that matches data of the brain signal by a preset percentage or greater, control an operation of the training apparatus based on whether the intention is recognized, and control playback of training content displayed on the training apparatus to correspond to the operation of the training apparatus.

### CROSS-REFERENCE TO RELATED APPLICATIONS

This application is a continuation-in-part of U.S. patent application Ser. No. 16/500,955, filed on Dec. 5, 2019, which is a national phase under 35 U.S.C. § 371 of PCT International Application No. PCT/KR2018/052223 filed on Mar. 30, 2018, which claims priority under 35 U.S.C. § 119 to Korean Patent Application No. 10-2017-0046691 filed on Apr. 11, 2017, in the Korean Intellectual Property Office, the disclosures of which are incorporated by reference herein in their entireties.

### TECHNICAL FIELD

The present disclosure relates to a simulator and a simulation system for brain training based on behavior modeling, and more particularly, to a simulator and a simulation system for brain training based on behavior modeling that recognizes an action intention of a user using brain signals, activates a training apparatus according to a recognized action intention, and maximizes rehabilitation training with stimulation driven inducement through neurofeedback.

### BACKGROUND ART

Rehabilitation therapy refers to a series of treatment processes performed by a patient who incurred damage to a body part due to a disease, an accident, a disaster or the like or who had major surgery and has entered into a period of convalescence to recover function of a damaged part or a weakened part.

Conventional rehabilitation therapies are performed by therapists, robots, electric stimulators, or the like, generally to patients unilaterally and passively, and uses a bottom-up type method, which is a rehabilitation exercise therapy of simple repetitions.

Recently, technology for rehabilitation therapy is rapidly developing, and currently at a transition period of being converted clinically. To remove the physical disabilities of patients with brain disease or disabled persons, there ultimately needs to be a combination of studies on physical therapies and treatment of brain plasticity.

Products for rehabilitating at home away from hospitals are being released after 2010, and intention detection technology for recognizing the movement intention of a person through a patch measuring electromyogram signals and assisting in rehabilitation is being introduced.

In lower limb rehabilitation robot technology, a brain-computer interface (BCI) is used with an over-ground type robot system. HAL, which was developed in 2009, is a first commercial exoskeleton-type walking assistance and rehabilitation robot.

Other prior art on rehabilitation training are disclosed in Korean Laid-Open Patent Publication No. 10-2014-0061170 through to Korean Registered Patent No. 10-1501524.

The prior art disclosed in Korean Laid-Open Patent Publication No. 10-2014-0061170 relates to providing information related to rehabilitation to patients inducing a patient rehabilitation intention and providing an active rehabilitation training suitable to patient state by continuously measuring bio-signals of a patent to monitor the patent state.

The prior art disclosed in Korean Registered Patent No. 10-1501524 relates to measuring brain signals of a patient and adjusting the time, intensity, or the like of rehabilitation exercise to encourage the patient to carry out rehabilitation exercise in an active environment.

### DISCLOSURE

### Technical Problem

Conventional rehabilitation therapy methods such as the above are, however, bottom-up type rehabilitation training methods for patients capable of physical movement, and has a disadvantage of being unsuitable for chronic patients experiencing a rehabilitation plateau as a complete sensor-motor looped rehabilitation is not achieved from a cerebral nerve perspective.

The prior art disclosed in Korean Laid-Open Patent Publication No. 10-2014-0061170 relates to a biofeedback rehabilitation training method using bio-signals such as electromyogram and foot pressure, but has the disadvantage of having a weak electromyogram signal or unsuitable to patients incapable of physical movement.

The prior art disclosed in Korean Registered Patent No. 10-1501524 uses brain signals to adjust the time, intensity, and the like of rehabilitation exercise to attempt an improvement of efficiency in rehabilitation training, but has the disadvantage of not being an optimal rehabilitation training system as recognition of user rehabilitation intention is merely comprised of recognition of a single action, and providing feedback on the state of rehabilitation training is impossible.

Accordingly, the present disclosure has been proposed to solve the general problems occurring from prior arts such as the above. An object of the present disclosure is to provide a simulator and a simulation system for brain training based on behavior modeling that recognizes user action intention using brain signals, operates the training apparatus according to the recognized intention, and maximizes rehabilitation training by stimulation driven motivation inducement through neurofeedback.

Another object of the present disclosure is to provide a simulator and a simulation system for brain training based on behavior modeling, which are applicable to various patient groups by allowing patients with degenerative brain diseases such as dementia or cerebral lesion disorders such as cerebral apoplexy to perform rehabilitation training to accelerate/enhance brain plasticity and strengthen brain signals.

Still another object of the present disclosure is to provide a simulator and a simulation system for brain training based on behavior modeling that performs rehabilitation training through brain signal based user intention recognition so that rehabilitation training is possible even to patients with degenerative brain diseases such as having weak electromyogram signals due to paralysis, dementia, or the like or cerebral lesion disorders such as cerebral apoplexy.

Still another object of the present disclosure is to provide a simulator and a simulation system for brain training based on behavior modeling that performs consecutive recognition of user intention so that rehabilitation training may be performed through various operations such as adjusting the level of difficulty (speed, intensity, time, etc.) of rehabilitation training or changing operation modes.

### Technical Solution

According to an embodiment of the present disclosure, a brain training simulator includes at least one memory, and at least one processor configured to acquire a brain signal of a user acting in a first action state based on a non-invasive brain activation measurement method, determine whether an intention of the user is recognized, by selecting preset intention data that matches data of the brain signal by a preset percentage or greater, control an operation of the training apparatus based on whether the intention is recognized, and control playback of training content displayed on the training apparatus to correspond to the operation of the training apparatus.

In addition, the acquired brain signal may include at least one of a metabolism brain signal related to exercise management of a cerebral cortex and information on an oxygen concentration of hemoglobin.

In addition, the determining may include, based on the selected intention data being intention data related to a second action state, determining that the intention is successfully recognized, and based on the selected intention data being intention data related to the first action state, determining that the intention is unsuccessfully recognized, and the controlling of the operation of the training apparatus may include, based on determining that the intention is successfully recognized, controlling the operation of the training apparatus for the second action state, and based on determining that the intention is unsuccessfully recognized, controlling the operation of the training apparatus for the first action state.

In addition, the at least one processor may be further configured to acquire training state information about an action of the user corresponding to the operation of the training apparatus, and the training state information may include at least one of a training distance, a training time, a number of times walking, a walking pattern, a number of times of intention recognition, a training distance based on an intention recognition, a training time based on the intention recognition, brain activation state information, biometric information about the user, a brain signal, and intention recognition information.

In addition, the at least one processor may be further configured to store training state information about the user in a profile corresponding to the user, and store the profile in an entire database of a patient group to which the user belongs.

In addition, the at least one processor may be further configured to generate an analysis data obtained by analyzing the data of the acquired brain signal of the user in real time, and generate diagnostic data on a disease of the user based on the generated analysis data and the entire database.

In addition, the at least one processor may be further configured to output at least one of whether the intention is recognized, training state information about an action of the user corresponding to the operation of the training apparatus, whether an operating mode of the training apparatus is changed, a message for immersion in training, and an alarm indicating an improvement of a training score.

In addition, the at least one processor may be further configured to output at least one of comprehensive information and information for preparing for dangerous situations, based on training state information about an action of the user corresponding to the operation of the training apparatus.

In addition, the determining may include inputting the data of the brain signal as input data to a recognition model, and determining whether the intention of the user is recognized, by acquiring, as output data, whether the intention of the user is recognized.

In addition, the recognition model may be trained based on an artificial intelligence-based machine learning method.

In addition, the controlling of the operation of the training apparatus may include, based on whether the intention of the user is recognized, controlling at least one of a speed, an intensity, and time of the training apparatus, a direction change within the training content, and an operation mode change of the training apparatus, while the training apparatus is in operation.

In addition, the acquiring may include, based on controlling the operation of the training apparatus for the second action state, acquiring the brain signal of the user based on the non-invasive brain activation measurement method, the determining may include, based on the selected intention data being intention data related to a third action state, determining that the intention is successfully recognized, and based on the selected intention data being intention data related to the second action state, determining that the intention is unsuccessfully recognized, and the controlling of the operation of the training apparatus may include, based on determining that the intention is successfully recognized, controlling the operation of the training apparatus for the third action state, and based on determining that the intention is unsuccessfully recognized, controlling the operation of the training apparatus for the second action state.

In addition, the training content may include at least one virtual avatar that operates based on whether the intention is recognized.

In addition, the training content may further include a virtual avatar related to an action with which the user is to be trained, and a virtual avatar related to the selected intention data.

According to an embodiment of the present disclosure, a brain training simulation system includes a brain training simulator configured to send training content to a training apparatus such that the training content is displayed by the training apparatus, acquire a brain signal of a user acting in a first action state based on a non-invasive brain activation measurement method, determine whether an intention of the user is recognized, by selecting preset intention data that matches data of the brain signal by a preset percentage or greater, control an operation of the training apparatus based on whether the intention is recognized, and control playback of the training content displayed by the training apparatus to correspond to the operation of the training apparatus, and a training apparatus configured to display the training content received from the brain training simulator and operate under control of the brain training simulator.

### Effect of Invention

According to the present disclosure, the present disclosure is advantageous for allowing rehabilitation training to be performed using various operations by recognizing user action intention with the use of brain signals, operating the rehabilitation training apparatus according to the recognized action intention, and changes the rehabilitation training speed or the operation mode in accordance with the consecutive recognition of user action intention.

Because rehabilitation training is performed based on user intention recognition using brain signals, the present disclosure is advantageous for being applicable to rehabilitation training of various patient groups by allowing even patients with degenerative brain diseases such as dementia or cerebral lesion disorders such as cerebral apoplexy to perform rehabilitation training to accelerate/enhance brain plasticity and strengthen brain signals.

Based on performing rehabilitation training through user intention recognition based on brain signals, the present disclosure is advantageous for being applicable even in the rehabilitation of patients with degenerative brain diseases such as having weak electromyogram signals due to physical paralysis, dementia, or the like or cerebral lesion disorders such as cerebral apoplexy.

### DESCRIPTION OF DRAWINGS

FIG. 1 is a diagram for describing a brain training simulation system according to an embodiment of the present disclosure;

FIG. 2A is a block diagram of a brain training simulator according to an embodiment of the present disclosure;

FIG. 2B is a block diagram illustrating another example of a brain training simulator according to an embodiment;

FIG. 3 is a block diagram specifying a control unit of a brain training simulator according to an embodiment of the present disclosure;

FIG. 4 is a diagram for describing an operation of a brain training simulator according to an embodiment of the present disclosure;

FIG. 5 is a diagram for describing the operation of a brain training simulation system, according to an embodiment of the present disclosure;

FIG. 6 is a diagram for describing a screen of a rehabilitation training content, according to an embodiment of the present disclosure;

FIG. 7 is a diagram for describing a monitoring screen of a rehabilitation training state, according to an embodiment of the present disclosure;

FIG. 8 is a diagram illustrating an image of a brain before rehabilitation training and after rehabilitation training based on user intention recognition, according to an embodiment of the present disclosure;

FIG. 9 is a diagram for comparing brain activation states before rehabilitation training and after rehabilitation training based on user intention recognition, according to an embodiment of the present disclosure;

FIG. 10 is a diagram illustrating a transition of intention recognition state, according to an embodiment of the present disclosure;

FIG. 11 is a diagram for describing a data collection protocol for each recognition model for user intention recognition, according to an embodiment of the present; and

FIG. 12 is a flowchart of a method of controlling a brain training simulator, according to an embodiment of the present disclosure.

### DETAILED DESCRIPTION OF THE EXEMPLARY EMBODIMENTS

Hereinafter, various embodiments will be described in detail with reference to the enclosed drawings. Embodiments disclosed in the present disclosure may be variously modified. A specific embodiment may be described in the drawings and described in detail in the detailed description. However, the specific embodiment disclosed in the enclosed drawing is merely to assist in the clear understanding of the various embodiments. Accordingly, the embodiments of the present disclosure are not limited in technical spirit by the specific embodiments disclosed in the enclosed drawings, and should be understood as including all equivalents included in the spirit of the present disclosure and technical scope, or alternatives therein.

Although terms including ordinal numbers such as first, second, or the like may be used to describe various elements, the elements are not to be limited by the terms described above. The terms are only used to distinguish one element from another element.

In the present specification, it is to be understood that the terms such as “comprise”, “include” or “have” are used herein to designate a presence of characteristic, number, step, operation, element, component, or a combination thereof, and not to preclude a presence or a possibility of adding one or more of other characteristics, numbers, stages, operations, elements, components or a combination thereof. It is to be understood that when a certain element is referred to as being “coupled to” or “connected to” another element, the element may be coupled to or connected to another element directly, but may also be understood as having a different element therebetween. On the other hand, it is to be understood that when a certain element is referred to as being “directly coupled to” or “directly connected to” another element, no other element is present therebetween.

The term such as “module” or “unit” is used to refer to an element that performs at least one function or operation. In addition, “module” or “unit” may perform afunction or operation implemented as hardware or software, or a combination of hardware and software. Further, except for a “module” or “unit” that needs to be realized in particular hardware or at least one processor, or to be performed in at least one control unit, a plurality of “modules” or a plurality of “units” may be integrated in at least one module. Unless otherwise defined specifically, a singular expression may encompass a plural expression.

In case it is determined that in describing embodiments, detailed description on function or configuration of related known technologies may unnecessarily confuse the gist of the disclosure, the detailed description will be summed or omitted. Meanwhile, the respective embodiments may be independently realized or operated, but also realized in combination or operated.

Hereinafter, a simulator and a simulation system for brain training based on behavior modeling according to an embodiment of the present disclosure will be described in detail with reference to the enclosed drawings.

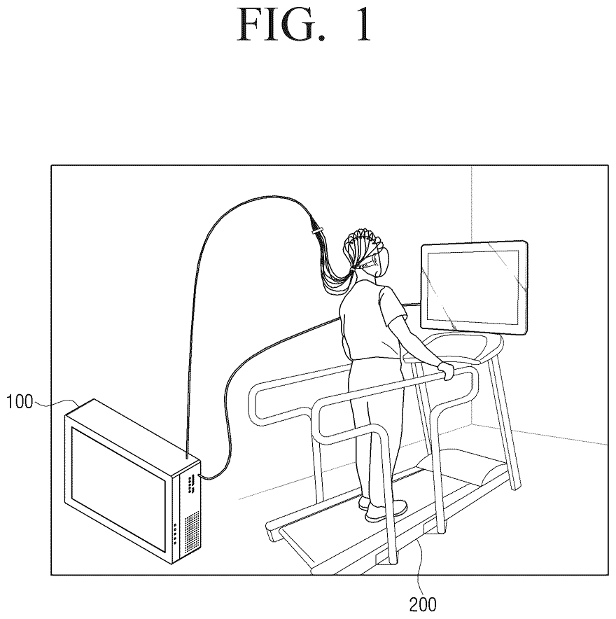

FIG. 1 is a diagram for describing a brain training simulation system according to an embodiment of the present disclosure.

Referring to FIG. 1, the brain training simulation system includes a brain training simulator 100 and a training apparatus 200. The brain training simulator 100 transmits training content to the training apparatus such that the training content is displayed by the training apparatus. The training apparatus 200 displays the training content received from the brain training simulator 100.

The brain training simulator 100 may acquire a brain signal of a user through an input device worn on the head of the user. For example, the brain training simulator 100 may acquire a brain signal of the user acting in a first action state, based on a non-invasive brain activation measurement method.

Noise components may be removed from the acquired brain signal through various preprocessing processes. For example, the brain signal may include at least one of metabolism brain signal related to exercise management of a cerebral cortex and information on an oxygen concentration of hemoglobin.

The brain training simulator 100 determines user intention based on the acquired brain signal data and preset intention data.

For example, the present intention data may be data accumulated by the artificial intelligence-based machine learning method. The preset intention data may be average data of normal people, average data of patients suffering from a specific disease, or accumulated personal data of a user performing brain training.

The brain training simulator 100 determines preset intention data matched with brain signal data as user intention. In the present disclosure, the meaning of matching may not only include an instance where the preset intention data and the acquired brain signal data is an exact match but also an instance where the match is over a certain percentage or higher. Further, in the case of determining user intention using artificial intelligence technology, the artificial intelligence technology may include determining user intention based on artificial intelligence based learning data.

The brain training simulator 100 may determine whether the user intention is recognized, by selecting preset intention data that matches the brain signal data by a preset percentage or greater.

The brain training simulator 100 controls the training apparatus 200 based on the determined user intention, and controls a playback of the training content to correspond to an operation of the training apparatus.

For example, the brain training simulator 100 controls the training apparatus 200 based on the determining of whether the user intention is recognized, and controls the playback of the training content to correspond to the operation of the training apparatus.

In addition, the brain training simulator 100 provides feedback for inducing brain activation of the user.

The training apparatus 200 may include a driving unit configured to move under control of the brain training simulator 100, and a display unit configured to display the received training content. Alternatively, the training apparatus 200 may be implemented such that a driving device and a display device are separate devices. The training apparatus 200 may display the received training content from the brain training simulator 100. Further, the training apparatus 200 may be operated under control of the brain training simulator 100, and may playback the training content. For example, the training apparatus 200 may include various rehabilitation apparatuses such as a treadmill, a training apparatus for walking assistance, a knee training apparatus, an ankle exercise apparatus, a robot-assisted training apparatus for walk rehabilitation, various rehabilitation apparatus including upper extremity rehabilitation training apparatus, robot and virtual reality driving apparatuses, or the like. The present disclosure describes about a brain training simulation system for rehabilitation purpose as an embodiment. However, as described above, the training apparatus 200 may be realized as various driving apparatuses.

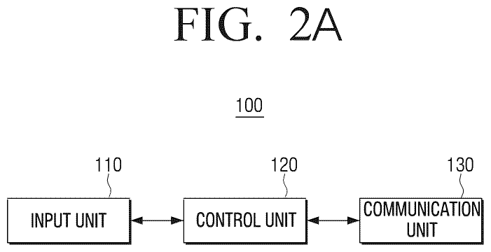

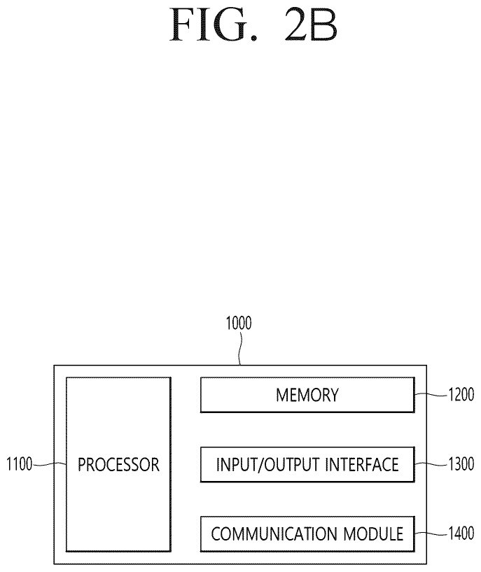

FIG. 2A is a block diagram of a brain training simulator according to an embodiment of the present disclosure.

Referring to FIG. 2A, the brain training simulator 100 includes an input unit 110, a control unit 120, and a communication unit 130.

The input unit 110 acquires a brain signal of a user based on a non-invasive brain activation measurement method. The input unit 110 may be placed on a head portion of the user. For example, the non-invasive brain activation measurement method may include methods such as an electroencephalogram (EEG), magnetoencephalogram (MEG), near-infrared spectroscopy (NIRS), magnetic resonance imaging (MRI), electrocorticogram (ECoG), and the like. Further, the acquired brain signal may include metabolism brain signal related to exercise management of a cerebral cortex or a signal on changes in oxygen concentration of hemoglobin.

The control unit 120 determines user intention based on the acquired brain signal data and the preset intention data. The preset intention data may be data accumulated by an artificial intelligence-based machine learning method. Further, the preset intention data may be the average brain signal data of normal people, the average data of patients suffering from a specific disease, or an accumulated personal data of a user performing brain training. For example, the preset intention data may be data on metabolism brain signal when a user is thought to be walking or a signal on changes in oxygen concentration of hemoglobin.

The control unit 120 determines preset intention data matched with data of acquired brain signals as user intention. Further, the control unit 120 controls an operation of the training apparatus 200 based on the determined user intention and controls a playback of training content to correspond to the operation of the training apparatus 200. For example, if the training apparatus 200 is a treadmill, and based on the control unit 120 determining that the user has a walking intention, the control unit may control the driving unit of the treadmill to operate to a walking speed and may perform the playback of the training content to match the driving speed of the treadmill. The control unit 120, if the user is a normal person, may control the driving speed of the treadmill to a walking speed of a normal person, and if the user has a brain disease or is disabled, may control the treadmill to operate at a speed significantly lower than the walking speed of a normal person.

The control unit 120 provides feedback for inducing brain activation to the user. For example, the feedback may include training state information, a message for immersion in training (for example, compliment, encouragement, etc.), an alarm indicating an improvement of a training score, and the like. Further, the control unit 120 may provide comprehensive information, information for dangerous situations, or the like based on training state information according to operation of the training apparatus 200 as training state feedback information. According to an embodiment, the training state information may include a training distance, a training time, a number of times walking, a walking pattern, a number of times of intention recognition, a training distance based on intention recognition, a training time based on intention recognition, brain activation state information, user biometric information, a brain signal, intention recognition information, or the like.

The control unit 120 acquires user training state information, and may determine whether the training mode is changed based on the acquired training state information. The control unit 120 may change the operation mode of the training content according to the changed training mode and induce brain activation to the user. For example, if the control unit 120 determines that a walking training based on user intention becomes accustomed to the user, the training mode may be changed to a faster paced walking training or running training. Further, the control unit 120 may provide motivation or stimulation to the user using the training content for inducing user brain activation.

The control unit 120 may control the training apparatus 200 based on a consecutive determination of user intention and the determined consecutive intention of user. The control unit 120 may determine the consecutive intention of the user after removing noise by performing a preprocessing process of the acquired brain signal data and through a wavelet transform. Further, the control unit 120 may control the speed, intensity, and time of the training apparatus 200 while the training apparatus 200 is in operation based on the consecutive intention of the user. Also, the control unit 120 may control a change in direction, a change in operation mode of the training apparatus, or the like within the training content while the training apparatus 200 is in operation based on the consecutive intention of the user. For example, conventional apparatuses may only perform signal operations such as a method of a user moving straight and having to first stop to change direction to the right and then changing direction to the right. However, because the brain training simulator 100 of the present disclosure determines user intention based on artificial intelligence based or accumulated data in real-time, determining user intention to change direction to the right while moving straight forward is possible. Therefore, if the training apparatus 200 is a treadmill displaying training content, the brain training simulator 100 changes direction of a screen playing back training content or may control an operation of the training apparatus 200 by determining user intention while operating at 1 km/h to 2 km/h.

The control unit 120 provides the user with an operation of a virtual avatar within the training content so that a user may model a behavior as feedback for inducing brain activation and may move the virtual avatar in accordance with the determined user intention.

Based on the user being a patient, the control unit 120 stores the acquired user training state information as a profile corresponding to the respective user, and may store the profile of respective users in an entire database of patient group which includes the user. Further, the control unit 120 generates analysis data by analyzing the data of the acquired brain signal of the user in real-time and may diagnose user disease based on the generated analysis data and the entire database. The database may be included in the storing unit of the brain training simulator 100, or may be included in a storing unit of a separate server.

The communication unit 130 transmits training content to the training apparatus for display, and the training content may be displayed in the training apparatus 200. In the case that the brain training simulation system may include a server including a database, the communication unit 130 performs communication with the server and transmits the acquired brain signal data, the generated analysis data or user profile to the server, and may receive the entire database of the patient group from the server. In certain cases, the brain training simulator 100 may transmit the acquired data of the patent to the server, and after the server diagnoses a disease of the user, may transmit the diagnosis result to the brain training simulator 100.

Although not illustrated in FIG. 2A, the brain training simulator 100 may further include an output unit (not shown). The output unit is configured to generate visual, auditory or tactile-sense related output and may output the above described feedback. The output unit may output the determined user intention, training state information, information on change of training mode, a message for focusing on training, an alarm for improving training score, a comprehensive information based on training state information according to operation of a training apparatus, information for dangerous situations, and the like. For example, the output unit may be realized as a display, a speaker, a buzzer, a haptic module, a light output unit.

The control unit 120 may include various elements (or modules).

FIG. 2B is a block diagram illustrating another example of a brain training simulator according to an embodiment.

Referring to FIG. 2B, a brain training simulator 1000 includes a processor 1100, a memory 1200, an input/output interface 1300, and a communication module 1400. For convenience of description, FIG. 2B illustrates only components related to the present disclosure. Thus, other general-purpose components other than the components illustrated in FIG. 2B may be further included in the brain training simulator 1000. In addition, it is apparent to those of skill in the art related to the present disclosure that the processor 1100, the memory 1200, the input/output interface 1300, and the communication module 1400 illustrated in FIG. 2B may be implemented as independent devices.

In addition, the processor 1100 of FIG. 2B may correspond to the control unit 120 of FIG. 2A, the input/output interface 1300 of FIG. 2B may correspond to the input unit 110 of FIG. 2A, and the communication module 1400 of FIG. 2B may correspond to the communication unit 130 of FIG. 2A. Thus, redundant descriptions of the processor 1100, the input/output interface 1300, and the communication module 1400 will be omitted.

The processor 1100 may process commands of a computer program by performing basic arithmetic, logic, and input/output operations. Here, the commands may be provided from the memory 1200 or an external device (not shown). In addition, the processor 1100 may control the overall operation of other components included in the brain training simulator 1000.

The processor 1100 may acquire a brain signal of the user acting in a first action state, based on a non-invasive brain activation measurement method.

Here, the acquired brain signal may include at least one of metabolism brain signal related to exercise management of a cerebral cortex and information on an oxygen concentration of hemoglobin.

In addition, the processor 1100 may determine whether an intention of the user is recognized, by selecting preset intention data that matches the brain signal data by a preset percentage or greater.

The processor 1100 may determine, based on the selected intention data being intention data related to a second action state, that the recognition of the intention is successful, and determine, based on the selected intention data being intention data related to the first action state, that the recognition of the intention is unsuccessful.

According to an embodiment of the present disclosure, based on the selected intention data being intention data related to the second action state, it is determined that the recognition of the intention is successful, and thus, brain activation of the user may be induced such that the user intends the second action state.

In addition, the processor 1100 may control the operation of the training apparatus based on whether the intention is recognized.

In addition, the processor 1100 controls, based on the intention recognition being successful, the operation of the training apparatus related to the second action state, and controls, based on the intention recognition being unsuccessful, the operation of the training apparatus related to the first action state.

According to an embodiment of the present disclosure, by controlling the operation of the training apparatus based on whether the intention is recognized, user-customized training may be performed by reflecting the intention of the user.

In addition, the processor 1100 may control the playback of training content displayed on the training apparatus to correspond to the operation of the training apparatus.

In addition, the processor 1100 may acquire training state information about the users action corresponding to the operation of the training apparatus. Here, the training state information may include at least one of a training distance, a training time, a number of times walking, a walking pattern, a number of times of intention recognition, a training distance based on an intention recognition, a training time based on the intention recognition, brain activation state information, user biometric information, a brain signal, and intention recognition information.

In addition, the processor 1100 may store the training state information of the user in a profile corresponding to the user, and store the profile in an entire database of a patient group including the user.

In addition, the processor 1100 may generate analysis data acquired by analyzing the acquired brain signal data of the user in real time, and generate diagnostic data regarding the users disease based on the generated analysis data and the entire database.

In addition, the processor 1100 may output at least one of whether the intention is recognized, training state information about the user's action corresponding to the operation of the training apparatus, whether the operating mode of the training apparatus is changed, a message for immersion in training, and an alarm indicating an improvement of a training score.

In addition, the processor 1100 may output at least one of comprehensive information and information for preparing for dangerous situations, based on training state information about the users action corresponding to the operation of the training apparatus.

In addition, the processor 1100 may determine whether the intention of the user is recognized, by inputting data of a brain signal to a recognition model as input data and acquiring, as output data, whether the intention is recognized.

Here, the recognition model may be trained based on an artificial intelligence-based machine learning method. For example, the recognition model may be a machine learning model.

The machine learning model refers to a statistical learning algorithm implemented based on the structure of a biological neural network, or a structure for executing the algorithm, in machine learning technology and cognitive science.

For example, the machine learning model may refer to a machine learning model that obtains a problem-solving ability by repeatedly adjusting the weights of synapses by nodes that are artificial neurons forming a network in combination with the synapses as in biological neural network, to learn such that an error between a correct output corresponding to a particular input and an inferred output is reduced. For example, the machine learning model may include an arbitrary probability model, a neural network model, etc., used in artificial intelligence learning methods, such as machine learning or deep learning.

For example, the machine learning model may be implemented as a multilayer perceptron (MLP) composed of multilayer nodes and connections therebetween. The machine learning model according to an embodiment of the present disclosure may be implemented by using one of various artificial neural network model structures including MLPs. For example, the machine learning model may include an input layer that receives an input signal or data from the outside, an output layer that outputs an output signal or data corresponding to the input data, and one or more hidden layers between the input layer and the output layer to receive a signal from the input layer, extract features, and deliver the features to the output layer. The output layer receives a signal or data from the hidden layer and outputs the signal or data to the outside.

In addition, based on whether the intention of the user is recognized, the processor 1100 may control at least one of a speed, an intensity, and time of the training apparatus, a direction change within the training content, and an operation mode change of the training apparatus while the training apparatus is in operation.

In addition, when controlling the operation of the training apparatus for the second action state, the processor 1100 may acquire a brain signal of the user based on a non-invasive brain activation measurement method, based on the selected intention data being the intention data related to a third action state, determine that the recognition of the intention is successful, based on the selected intention data being the intention data related to the second action state, determine that the recognition of the intention is unsuccessful, based on determining that the recognition of the intention is successful, control the operation of the training apparatus for the third action state, and based on determining that the recognition of the intention is unsuccessful, control the operation of the training apparatus for the second action state.

According to an embodiment of the present disclosure, by controlling the operation of the training apparatus for the first and second action states as well as the third action state, it is possible to provide rehabilitation training for various actions, such as adjusting the level of difficulty (for example, speed, intensity, time, etc.) or changing the operating mode, through continuous recognition of the intention of the user.

In addition, the training content may include at least one virtual avatar that operates based on whether the intention is recognized.

In addition, the training content may include a virtual avatar related to an action with which the user is to be trained, and a virtual avatar related to the selected intention data. For example, when the training starts, the virtual avatar related to the action with which the user is to be trained may be a virtual avatar related to a second action, and the virtual avatar related to the selected intention data may be a virtual avatar related to a first action. As another example, when the recognition of the intention is unsuccessful, the virtual avatar related to the action with which the user is to be trained may be a virtual avatar related to the second action, and the virtual avatar related to the selected intention data may be a virtual avatar related to the first action. As another example, when the recognition of the intention is successful, the virtual avatar related to the action with which the user is to be trained may be a virtual avatar related to a third action, and the virtual avatar related to the selected intention data may be a virtual avatar related to the second action.

According to an embodiment of the present disclosure, by controlling the playback of the training content including the virtual avatar related to the action with which the user is to be trained and the virtual avatar related to the selected intention data, a target action desired by the user may be intuitively suggested through the virtual avatar related to the action with which the user is to be trained, and the user may be induced to intuitively grasp his or her current intention through the virtual avatar related to the selected intention data, and easily achieve behavior modeling such as a motor imagery or observing an action.

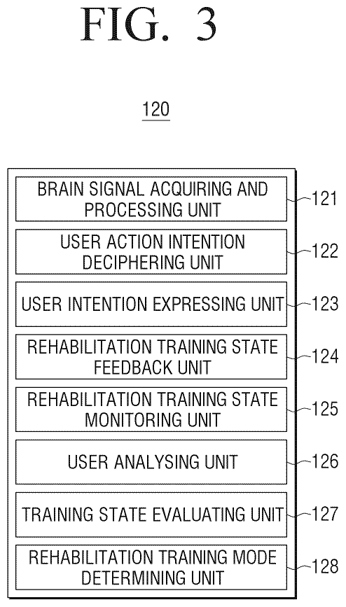

Detailed examples in which the processor 1100 according to an embodiment operates will be described with reference to FIGS. 3 to 12.

The processor 1100 may be implemented as an array of a plurality of logic gates, or may be implemented as a combination of a general-purpose microprocessor and a memory storing a program executable by the microprocessor. For example, the processor 1100 may include a general-purpose processor, a central processing unit (CPU), a microprocessor, a digital signal processor (DSP), a controller, a microcontroller, a state machine, etc. In some environments, the processor 1100 may include an application-specific integrated circuit (ASIC), a programmable logic device (PLD), afield-programmable gate array (FPGA), etc. For example, the processor 1100 may refer to a combination of processing devices, such as a combination of a DSP and a microprocessor, a combination of a plurality of microprocessors, a combination of one or more microprocessors combined with a DSP core, or a combination of any other such configurations.

The memory 1200 may include any non-transitory computer-readable recording medium. For example, the memory 1200 may include a permanent mass storage device, such as random-access memory (RAM), read-only memory (ROM), a disk drive, a solid-state drive (SSD), or flash memory. As another example, the permanent mass storage device, such as ROM, an SSD, flash memory, or a disk drive, may be a permanent storage device separate from the memory. Also, the memory 1200 may store an operating system (OS) and at least one piece of program code (e.g., code for the processor 1100 to perform an operation to be described below with reference to FIGS. 3 to 12).

These software components may be loaded from a computer-readable recording medium separate from the memory 1200. The separate computer-readable recording medium may be a recording medium that may be directly connected to the brain training simulator 1000, and may include, for example, a computer-readable recording medium, such as a floppy drive, a disk, a tape, a digital video disc (DVD)/compact disc ROM (CD-ROM) drive, or a memory card. Alternatively, the software components may be loaded into the memory 1200 through the communication module 1400 rather than a computer-readable recording medium. For example, at least one program may be loaded to the memory 1200 based on a computer program (for example, a computer program for the processor 1100 to perform an operation to be described below with reference to FIGS. 3 to 12) installed by files provided by developers or a file distribution system that provides an installation file of an application, through the communication module 1400.

The input/output interface 1300 may be a unit for an interface with a device (e.g., a keyboard or a mouse) for input or output that may be connected to the brain training simulator 1000 or included in the brain training simulator 1000. The input/output interface 1300 may be implemented separately from the processor 1100, but the present disclosure is not limited thereto, and the input/output interface 1300 may be implemented to be included in the processor 1100.

The communication module 1400 may provide a configuration or function for a server and the brain training simulator 1000 to communicate with each other through a network. In addition, the communication module 1400 may provide a configuration or function for the brain training simulator 1000 to communicate with another external device. For example, a control signal, a command, data, and the like provided under control of the processor 1100 may be transmitted to the server and/or an external device through the communication module 1400 and a network.

Meanwhile, although not illustrated in FIG. 2A, the brain training may further include a display device. For example, the display device may be implemented as a touch screen. Alternatively, the brain training simulator 1000 may be connected to an independent display device by a wired or wireless communication method to transmit and receive data to and from the display device. For example, training content and the like may be provided through the display device.

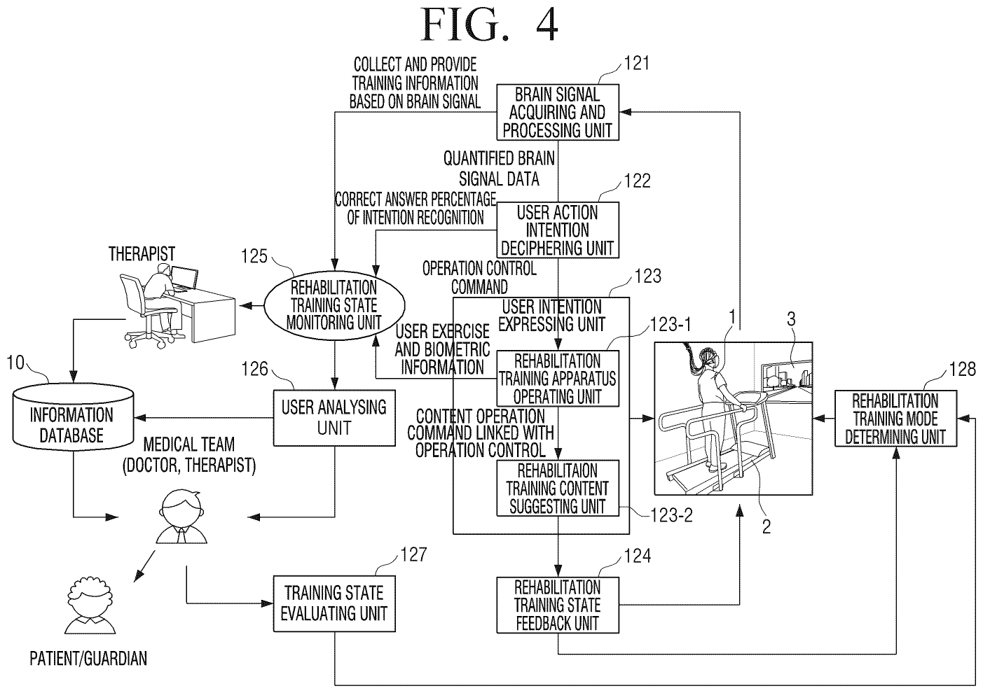

FIG. 3 is a block diagram specifying a control unit of a brain training simulator according to an embodiment of the present disclosure, and FIG. 4 is a diagram for describing an operation of a brain training simulator, according to an embodiment of the present disclosure.

The control unit 120 may include a brain signal acquiring and processing unit 121, a user action intention deciphering unit 122, a user intention expressing unit 123, a rehabilitation training state feedback unit 124, a rehabilitation training state monitoring unit 125, a user analysis unit 126, a training state evaluating unit 127, and a rehabilitation training mode determining unit 128.

Meanwhile, as described above, the brain signal acquiring and processing unit 121, the user action intention deciphering unit 122, the user intention expressing unit 123, the rehabilitation training state feedback unit 124, the rehabilitation training state monitoring unit 125, the user analysis unit 126, the training state evaluating unit 127, and the rehabilitation training mode determining unit 128 of FIG. 3 may be included in the processor 1100 of FIG. 2B. Detailed examples of operations of the processor 1100 will be described as operations of the brain signal acquiring and processing unit 121, the user action intention deciphering unit 122, the user intention expressing unit 123, the rehabilitation training state feedback unit 124, the rehabilitation training state monitoring unit 125, the user analysis unit 126, the training state evaluating unit 127, and the rehabilitation training mode determining unit 128.

The brain signal acquiring and processing unit 121 may acquire and process brain signals of a user (patient) 1 by a non-invasive brain activation measurement method. The quantitatively processed brain signal data is sent to the user action intention deciphering unit 122, and acquired training information based on brain signals may be sent to the rehabilitation training state monitoring unit 125. For example, brain signals may be measured by methods such as an electroencephalogram (EEG), magnetoencephalogram (MEG), near-infrared spectroscopy (NIRS), magnetic resonance imaging (MRI), electrocorticogram (ECoG), and the like.

The user action intention deciphering unit 122 may recognize an action intention of the user based on brain signal data processed by the brain signal acquiring and processing unit 121.

The user action intention deciphering unit 122 may determine whether the intention of the user is recognized, by selecting preset intention data that matches the brain signal data by a preset percentage or greater. The preset percentage may be set by an input from the user or a developer, but is not limited thereto.

The user action intention deciphering unit 122 removes noise through a preprocessing method (hemodynamic response function (HRF)) and a wavelet transform of the acquired brain signal data, and recognizes user action intention through an artificial intelligence based machine learning method (SVM; support vector machine, DNN; deep neural network, GP; genetic programming).

The user action intention deciphering unit 122 may provide information on number of user action intention recognitions to the rehabilitation training state monitoring unit 125.

The user intention expressing unit 123 may then operate the training apparatus 200 according to the action intention of the user recognized by the user action intention deciphering unit 122. The user intention expressing unit 123 may control the operation of the training apparatus based on whether the intention is recognized. The training apparatus 200 may be realized as various rehabilitation apparatuses such as a treadmill, a training apparatus for walking assistance, a knee training apparatus, an ankle exercise apparatus, a robot-assisted training apparatus for walk rehabilitation, various rehabilitation apparatus including upper extremity rehabilitation training apparatus, robot and virtual reality driving apparatuses, or the like, but for convenience of description, a treadmill will be described as being used as the training apparatus for rehabilitation training in the present disclosure.

The user intention expressing unit 123 operates the training apparatus 200 according to the recognized action intention of the user, and may include an a rehabilitation training apparatus operating unit 123-1 acquiring user exercise information according to the operation of the training apparatus 200 and a rehabilitation training content suggesting unit 123-2 providing the acquired user exercise information to the user through rehabilitation training content.

The user exercise information may include at least one of a training distance, a training time, a number of times walking, a walking pattern, a rehabilitation training distance based on intention recognition, and a rehabilitation training time based on intention recognition.

The rehabilitation training apparatus operating unit 123-1 may control the level of difficulty (speed, intensity, time, etc.) change in operation mode of the training apparatus 200 based on consecutive recognition of user intention when operating the training apparatus 200, may acquire user rehabilitation training information (exercise information) of a user according to an operation of the training apparatus 200 and send the user rehabilitation training information to the rehabilitation training state monitoring unit 125.

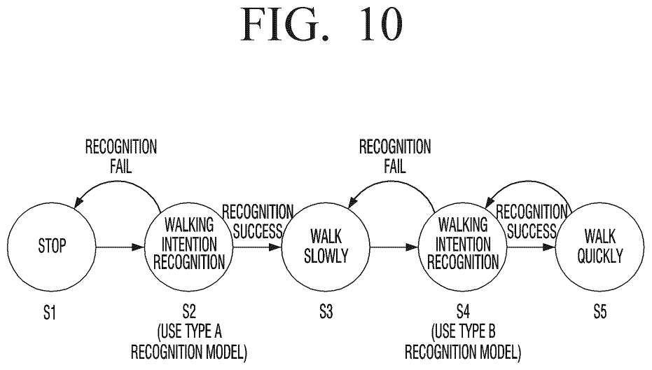

The rehabilitation training apparatus operating unit 123-1 may control an operation of the training apparatus 200 according to the consecutive recognition of the intention of the user based on an intention recognition state transition diagram as in FIG. 10. Transition of intention recognition state transitions in the order of a stop state S1, a walking intention recognition state S2, a walk slowly state S3, a walking intention recognition state S4, and a walk quickly state S5, but may transition to the next step if intention recognition is a success or may transition back to a previous stage if intention recognition is a fail. For example, the first action state may be the stop state S1, the second action state may be the walk slowly state S3, and the third action state may be the walk quickly state S5.

The rehabilitation training content suggesting unit 123-2 operates a virtual avatar to induce the user to easily achieve behavior modeling such as a motor imagery or observing an action, operates the virtual avatar according to the rehabilitation intention of the user, and may provide rehabilitation training content for improving cognitive ability. The rehabilitation training content may include at least one of a message for focusing on rehabilitation training, a text or voice on the training state, an electric tactile-sense for inducing compensation of brain activation according to improvement in training score.

In addition, the rehabilitation training state feedback unit 124 may suggest neurofeedback for inducing brain activation according to the rehabilitation training content suggested by the user intention expressing unit 123.

In addition, the rehabilitation training state monitoring unit 125 may monitor the training state information acquired respectively from the brain signal acquiring and processing unit 121, the user action intention deciphering unit 122, the user intention expressing unit 123, and the user intention expressing unit 123 in real-time.

For example, the rehabilitation training state monitoring unit 125 may feedback the exercise information, the biometric information, the brain signal, and the intention recognition information (number of times of intention recognition) of the user according to operation of the training apparatus as information for preparing a comprehensive assessment and preparing for dangerous situations.

The rehabilitation training state monitoring unit 125 may feedback training information based on brain signal, rehabilitation training distance, rehabilitation training time, rehabilitation training distance based on intention recognition, rehabilitation training time based on intention recognition, brain activation state as evaluation information for diagnosing and treating the user.

The analysis unit 126 may then analyze the training state information monitored by the rehabilitation training state monitoring unit 125 and may provide a determination information for evaluating training state.

Here, the determination information for evaluating training state is information for the physician to diagnose and treat, and thus may be seen as expert information.

An information database 10 stores the user rehabilitation training information acquired from the rehabilitation training state monitoring unit 125 in an individual profile, and the rehabilitation training information may be stored in an entire rehabilitation database classified by patient groups. For example, the information database 10 includes individual profiles storing individual rehabilitation information of users currently undergoing rehabilitation and the individual rehabilitation information of users stored in the individual profiles, and may include an entire rehabilitation database stored with the entire rehabilitation information in which the rehabilitation information of multiple rehabilitation patients is classified into patient groups. The information database 10 may be stored in the brain training simulator 100 or in a separate server (not shown).

The training state evaluating unit 127 stores the user training state information provided by the user analysis unit 126 and a result of analysis by a therapist through the rehabilitation training state monitoring unit 125 in real-time, and suggests feedback for changing of the rehabilitation training mode based on the determination result by determining whether to change the rehabilitation operation mode based on the above.

The training state evaluating unit 127 analyzes brain signals acquired during rehabilitation training in real time to use in the diagnosis of the user and in the early detection of a disease, uses the information accumulated in the information database 10 to evaluate the rehabilitation effect of the user currently undergoing rehabilitation, and compares the current rehabilitation training data acquired in real-time with the rehabilitation training information accumulated in the information database 10 to feedback a training protocol suitable to the current user.

In addition, the rehabilitation training mode determining unit 128 determines the rehabilitation training mode based on the neurofeedback information suggested by the a training state evaluating unit 127 and the rehabilitation training state feedback unit 124 to operate the training apparatus 200.

Each element of the control unit 120 may be realized as a software within the control unit 120 or configured as a hardware module. Alternatively, each element of the control unit 120 is realized in an individual hardware component, and an integration of each element may be realized as the control unit 120.

A detailed description of an operation of a brain signal simulation system based on behavior modeling according to an exemplary embodiment of the present disclosure as configured herein is as follows.

In the present disclosure, rehabilitation training is achieved based on behavior modeling. Behavior modeling refers to learning new behavior by observing action, motor imagery, and motor imagery based on observing action. The present disclosure, which applies the above, uses brain signals of a user (patient) to change the speed or operation mode of the training apparatus to perform rehabilitation training based on recognizing action intention of the user and the recognized action intention, and observes the user brain signal to define the consecutive recognition of user action intention as behavior modeling. The user action intention refers to reacting to content provided virtually, and may be confirmed through brain signal analysis.

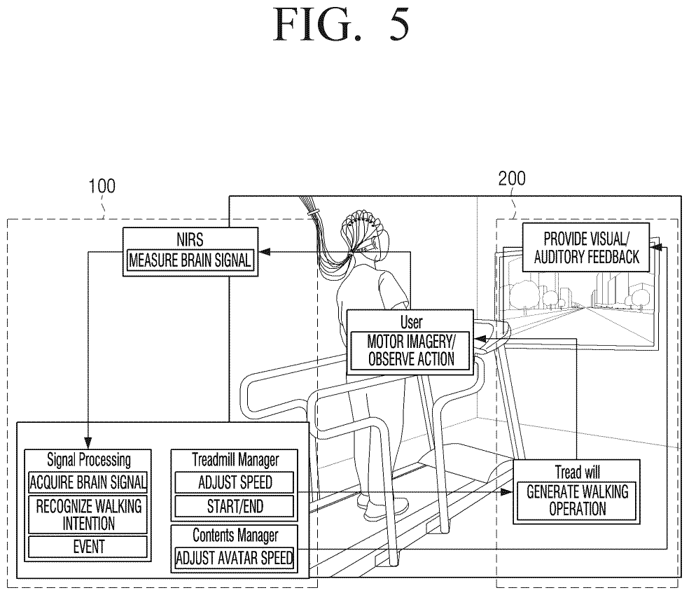

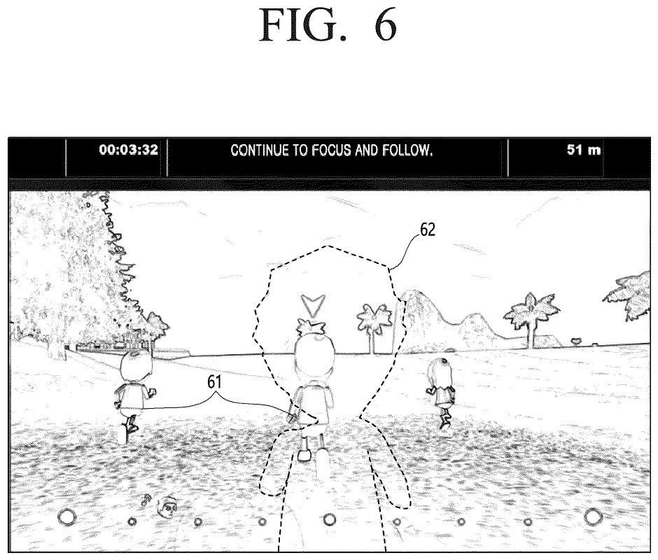

Based on a user (patient) 1 subject to rehabilitation in a prepared state for rehabilitation training using the rehabilitation training simulator as illustrated in FIG. 5, the brain training simulator 100 may inform user of rehabilitation schedule, method, or the like through the display unit of the training apparatus 200. The brain training simulator 100, as disclosed in FIG. 6, uses content such as an avatar to visually show an initial walking action (for example, an avatar walking action of 0.7 km/h), and by visually showing the content of an avatar running first, induces imagination to the user to follow the avatar. As described above, the training apparatus 200 includes a display unit and may display rehabilitation content. Alternatively, the display unit for displaying rehabilitation content may be realized separately from the training apparatus 200.

The brain training simulator 100 may control playback of training content including a virtual avatar related to an action with which the user is to be trained. As illustrated in FIG. 6, a virtual avatar 61 related to an action with which the user is to be trained may visually show the action with which the user is to be trained (for example, a walking action at 0.7 km/h).

In addition, the brain training simulator 100 may control the playback of the training content including a virtual avatar related to the selected intention data. As illustrated in FIG. 6, a virtual avatar 62 related to the selected intention data may visually show an action (e.g., a stop action) related to the selected intention data.

In addition, when the selected intention data is intention data related to the first action state, the virtual avatar 62 may visually show the first action state (e.g., a stop action). In addition, the virtual avatar 61 related to the action with which the user is to be trained may visually show the action with which the user is to be trained (e.g., a walking action at 0.7 km/h).

FIG. 5 is a diagram for describing the operation of a brain training simulation system, according to an embodiment of the present disclosure.

After the user sees the avatar displayed in the display unit and responds, the brain signal acquiring and processing unit 121 measures the brain signal of the user.

The rehabilitation training simulator as in FIG. 5 uses a treadmill of the training apparatus 200, and the treadmill manager refers to the rehabilitation training apparatus operating unit 123-1 of FIG. 4. The content manager indicates the rehabilitation training content suggesting unit 123-2 of FIG. 4, and signal processing refers to the brain signal acquiring and processing unit 121 and the user action intention deciphering unit 122 of FIG. 4.

Brain signals may be measured through methods such as an electroencephalogram (EEG), a magnetoencephalogram (MEG), a near infrared spectroscopy (NIRS), a magnetic resonance imaging (MRI), an electrocorticogram (ECoG), and the like.

According to an embodiment, the brain training simulator 100 uses a near infrared spectroscopy (NIRS) during user motor imagery or observing action to acquire metabolism brain signal related to exercise management of a cerebral cortex or an oxygen concentration of hemoglobin as user brain signal.

The brain training simulator 100 may provide the acquired brain signal to the rehabilitation training state monitoring unit 125 as brain signal based training information. Further, the acquired brain signal may be sent to the user action intention deciphering unit 122 processed as quantified brain signal data.

The user action intention deciphering unit 122 may remove noise elements such as user breathing, blood circulation and movement by processing the quantified brain signal data processed from the brain signal acquiring and processing unit 121 through various preprocessing methods (hemodynamic response function (HRF)) and wavelet transform. Further, the user action intention deciphering unit 122 may process the noise element removed brain signal through an artificial intelligence based machine learning method (SVM; support vector machine, DNN; deep neural network, GP; genetic programming) and may recognize user action intention with the result signal.

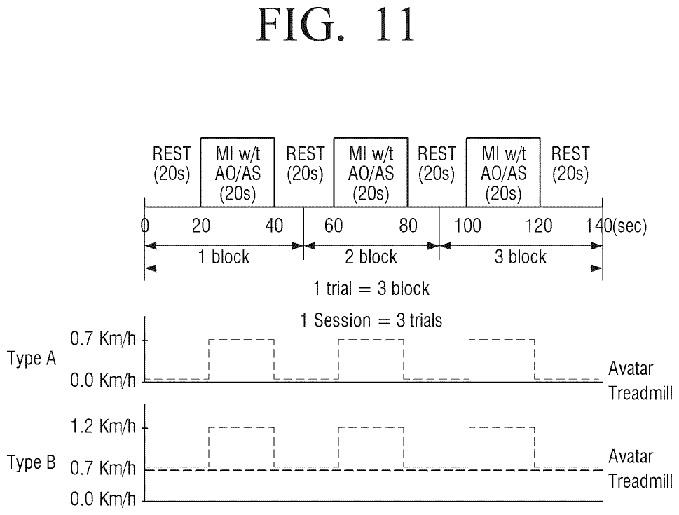

The user action intention deciphering unit 122 may recognize user action intention by using a recognition model using a training data collection protocol such as first recognition model (Type A) of FIG. 11 for the recognition of user action intention.

The user action intention deciphering unit 122 counts recognition of user action intention occurring normally as number of times of successful intention recognition, sends to the rehabilitation training state monitoring unit 125, and at the same time may provide an operation control command according to initial walking action to the user intention expressing unit 123. Based on recognition of user action intention failing, the user action intention deciphering unit 122 may perform the previous process again after resting for a predetermined time (for example, 30 seconds) and recognize the user action intention.

The rehabilitation training apparatus operating unit 123-1 of the user intention expressing unit 123 may operate the treadmill 2 to an initial walking action (0.7 km/h) based the initial walking action control command being sent by user action intention recognition. The rehabilitation training content suggesting unit 123-2 may use voice, text, or the like to provide a message concerning a compliment or encouragement. Further, the rehabilitation training content suggesting unit 123-2 may induce an imagination of continuously following.

The rehabilitation training content suggesting unit 123-2 uses the rehabilitation training content (avatar) after a predetermined time has passed to visually show the next walking action (for example, avatar walking action of 1.2 km/h), and visually shows the content of an avatar running faster to induce imagination so as to follow the avatar.

Based on the user seeing the avatar displayed in the display unit and reacting, the brain signal acquiring and processing unit 121 acquires user brain signal.

The user action intention deciphering unit 122 processes the quantified brain signal data processed in the brain signal acquiring and processing unit 121 and recognizes user action intention with the result signal. The user action intention deciphering unit 122 may recognizes user action intention by using a recognition model using a training data collection protocol such as Type B of FIG. 11 for recognition of user action intention.

The user action intention deciphering unit 122 counts recognition of user action intention occurring normally as number of times of successful intention recognition, sends to the rehabilitation training state monitoring unit 125, and at the same time may provide an operation control command according to the next walking action to the user intention expressing unit 123. Based on recognition of user action intention failing, the user action intention deciphering unit 122 may transition to the previous process after resting for a predetermined time (for example, 30 seconds) to walk the avatar in an initial operation mode and show a message to walk slowly to revert the user rehabilitation operation back to a previous stage.

The present disclosure performs recognition of not a single action but consecutive action intention based on recognition of user rehabilitation intention, and may perform rehabilitation training through various operations such as adjusting the level of difficulty (speed, intensity, time, etc.) of rehabilitation training, changing the operation mode, and the like.

FIG. 10 is a diagram illustrating a transition of intention recognition state, according to an embodiment of the present disclosure.

The brain training simulator 100 suggests rehabilitation training content through an avatar in an initial state of a stop state S1, and in a user walking intention recognition state S2 which is the next state, may recognize user walking intention using a first recognition model (Type A) as in FIG. 11. The brain training simulator 100 may transition to a stop state S1 based on a recognition fail occurring, and may transition to a walk slowly state S3 based on recognition success. The brain training simulator 100 transitions to a walking intention recognition state S4 again after a predetermined time from a state of having transitioned to a walk slowly state, and may recognize walking intention by using a second recognition model (Type B) as in FIG. 11. The brain training simulator 100 may transition to the walk slowly state S3 which is the previous state, and may transition to a walk quickly state S5 which is the next state based on recognition success.

The state transition described above is one embodiment to describe the state transition according to consecutive action intention of the present disclosure, but the present disclosure is not limited thereto, and including both the changing the order of the state transition or state transition that changes content is obvious to those having ordinary skill in the relevant field.

The present disclosure performs recognition of consecutive action intention and may perform rehabilitation training through various operations such as adjusting the level of difficulty (speed, intensity, time, etc.) of rehabilitation training, changing the operation mode, and the like.

The rehabilitation training state feedback unit 124 communicates with the user intention expressing unit 123, and may suggest visual/auditory stimulation by a message for compliment/encouragement based on rehabilitation training state, training speed, or the like in text or voice form through the display unit of the training apparatus 200. The training apparatus 200 may further an audio output device such as a speaker or a tactile-sense output device such as a haptic module or motor. The rehabilitation training state feedback unit 124 performs the role of suggesting neurofeedback for inducing brain activation according to improvements in rehabilitation training score, and may make possible rehabilitation training for the acceleration/enhancement of brain plasticity and strengthening of brain signals.

The user action intention deciphering unit 122 may provide the number of times of successful intention recognition each time intention recognition is performed normally during rehabilitation training to the rehabilitation training state monitoring unit 125 in real-time.

The rehabilitation training apparatus operating unit 123-1 of the user intention expressing unit 123 measures user exercise information from the beginning of rehabilitation training and may send to the rehabilitation training state monitoring unit 125 in real-time.

For example, user exercise information includes rehabilitation training distance, rehabilitation training time, number of times of walking, walking pattern, rehabilitation training distance based on intention recognition, rehabilitation training time based on intention recognition, and the like. Rehabilitation training distance, rehabilitation training time, and the like may be obtained through the training apparatus, the walking pattern may be obtained using sensors such as foot pressure sensor, inertial measurement unit (IMU) sensor, photo sensor, and infrared ray (IR) sensor, and the level of training focus may be obtained from result information of user intention recognition (number of times of success or success rate of intention recognition). Based on intention recognition, the rehabilitation training distance and the rehabilitation training time may also be easily extracted based on intention recognition information. The above described user exercise information and the like may be output through the output unit of the brain training simulator 100.

A therapist may observe the rehabilitation training state of a user (patient) in real-time based on information processed in the rehabilitation training state monitoring unit 125 and output through the output unit. The therapist may respond to emergency situations in real-time while performing rehabilitation training by monitoring the output information.

The therapist may additionally prepare an assessment on the patient state while at the same time monitoring the user rehabilitation training state in real-time. For example, after preparing a qualitative and quantitative assessment on walking quality which is not provided in the real-time monitoring, the assessment may be stored in the database. The patient state of the day is recorded after completing rehabilitation training in actual clinical practices.

The rehabilitation training information observed in real-time is stored in individual patient profiles, and may be analyzed through the user analysis unit 126.

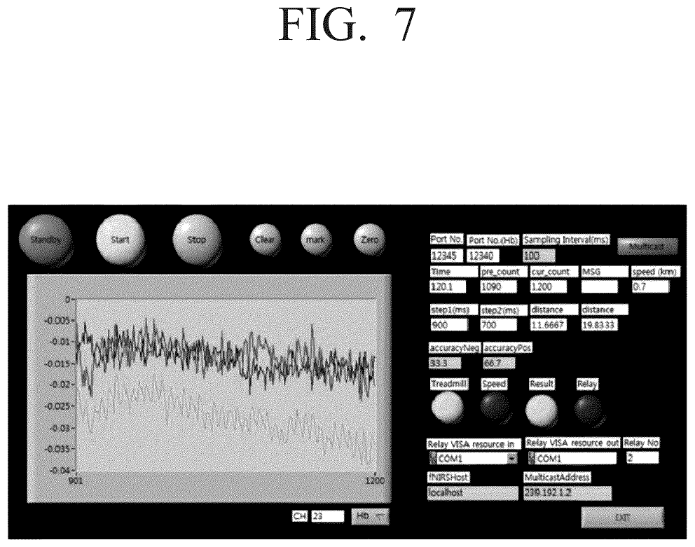

For example, the user analysis unit 126 analyzes the training state information monitored by the rehabilitation training state monitoring unit 125 and may provide the same as a determining information for training state evaluation. The determining information for evaluating training state is information for the physician to diagnose and treat, and thus may be seen as expert information. FIG. 7 is a screen example showing the result of the analyzed rehabilitation training state information.

During rehabilitation training, a medical team (physician, therapist) analyzes the rehabilitation training information analyzed in the user analysis unit 126 and the entire rehabilitation training information of rehabilitation patients per patient group accumulated in the information database 10 in real-time, and may perform diagnosis of a patient and early detection of a disease. In particular, the medical team may use the rehabilitation training information of patient groups accumulated for a long period and may perform clinical management such as evaluating the effect of rehabilitation on the respective patient. In the case of a new patient, the medical team may compare the current rehabilitation training data acquired in real-time with the rehabilitation training information of patient groups accumulated in the information database 10, and may suggest a training protocol suitable to a respective patient to perform an effective rehabilitation training. The neurofeedback information according to the training state evaluation of the medical team may be sent to the rehabilitation training mode determining unit 128.

For example, in a situation where rehabilitation is occurring in real-time, the medical team analyzes the rehabilitation training state of the patient to determine whether to change the rehabilitation operation mode, and sends the determination result to the rehabilitation training mode determining unit 128. That is, rehabilitation training state is analyzed in real-time during rehabilitation training, and determinations such as whether or not raising the rehabilitation training intensity of the respective patient is beneficial or lowering the same would be beneficial, whether or not maintaining the current state is beneficial, or the like may be made and provided to the rehabilitation training mode determining unit 128 via online or the like in real-time.

Based on machine learning based artificial intelligence technology being applied to the brain training simulator 100, the monitoring and analysis of the medical team described above may be performed by the brain training simulator 100.

The rehabilitation training mode determining unit 128 determines rehabilitation training mode based on the rehabilitation training information feedback by the rehabilitation training state feedback unit 124 and the analysis information neurally feedback by the training state evaluating unit 127 in real-time, maintains the current state or changes rehabilitation training mode according to the determined rehabilitation training mode, and may perform the optimum rehabilitation training operation.

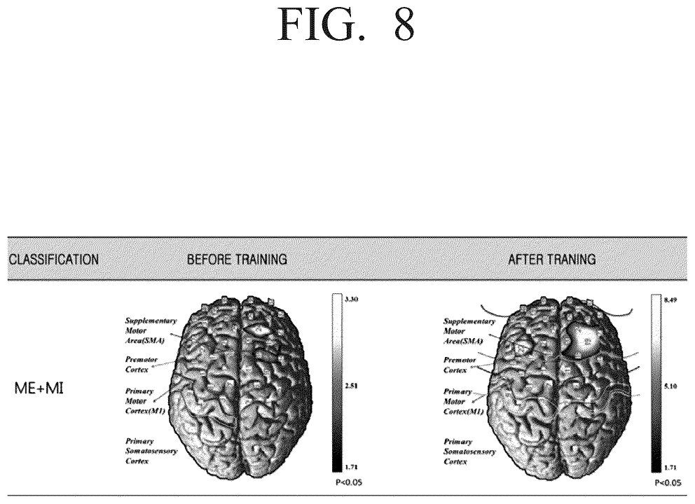

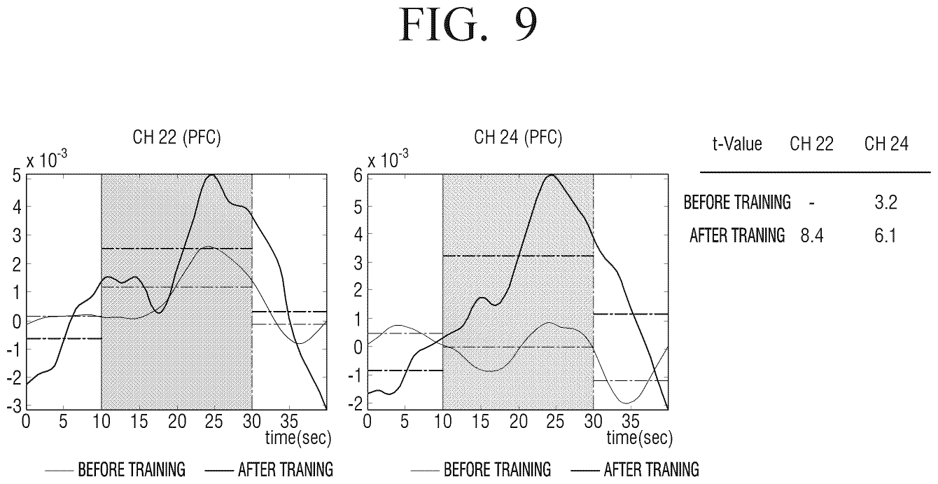

The test results of rehabilitation training system for accelerating brain plasticity according to the present disclosure is illustrated in FIGS. 8 and 9. FIG. 8 is a view illustrating an image of a brain before rehabilitation training and after rehabilitation training based on user intention recognition according to an embodiment of the present disclosure, and FIG. 9 is a view comparing a brain activation state before rehabilitation training and after rehabilitation training based on user intention recognition according to an embodiment of the present disclosure.

The left image or graph in FIGS. 8 and 9 is the result of performing motor imagery (MI) through observing action during motor execution (ME) prior to training, and the right image or graph shows the result of performing motor imagery (MI) through observing action during motor execution (ME) after training reflecting user intent.

As illustrated in FIG. 8, test results show significant activation in the frontal lobe responsible for physical movement according to cognitive function and focus, planning, thoughts and determinations during rehabilitation training on a treadmill that reflects user intention.

As illustrated in FIG. 9, activation was indicated at the 24-channel of the frontal lobe prior to training, and that activation was also confirmed at the 22-channel other than the 24-channel after training. It is apparent through FIGS. 8 and 9 that brain activation state increased in certain regions after training, and that oxidized hemoglobin also increased compared to before training. As a result, rehabilitation training that recognize user intention and is performed based on the recognized user intention may provide various patient groups with the optimum rehabilitation training.

Various embodiments of a braining training simulator and simulation system have been described above. A control method of a brain training simulator will be described below.

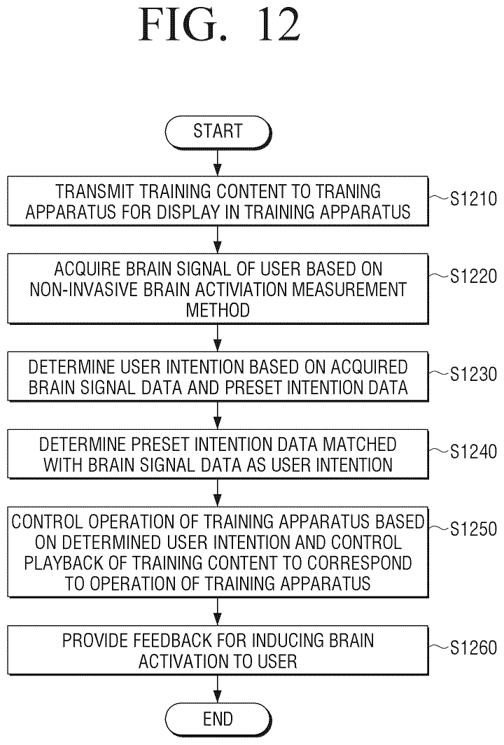

FIG. 12 is a flowchart of a method of controlling a brain training simulator, according to an embodiment of the present disclosure.

Referring to FIG. 12, the brain training simulator sends the training content to the training apparatus to display in the training apparatus (S1210). For example, the training apparatus may include a treadmill, a training apparatus for walking assistance, a knee training apparatus, an ankle exercise apparatus, a robot-assisted training apparatus for walk rehabilitation, various rehabilitation apparatus including upper extremity rehabilitation training apparatus, robot and virtual reality driving apparatuses, or the like. The training apparatus may include a display unit for displaying received training content. Further, the brain training simulation system may include a display apparatus separate from the training apparatus.

The brain training simulator acquires user brain signal based on a non-invasive brain activation measurement method (S1220). For example, the brain training simulator may acquire a brain signal of the user acting in a first action state, based on a non-invasive brain activation measurement method.

Here, the non-invasive brain activation measurement method may include methods such as an electroencephalogram (EEG), magnetoencephalogram (MEG), near-infrared spectroscopy (NIRS), magnetic resonance imaging (MRI), electrocorticogram (ECoG), and the like.

The acquired brain signal may include metabolism brain signal related to exercise management of a cerebral cortex or a signal on changes in oxygen concentration of hemoglobin.

The brain training simulator determines user intention based on the data of the obtained brain signal and the data of the present intention data (S1230). For example, the present intention data may be data accumulated by the artificial intelligence-based machine learning method. The preset intention data may be average data of normal people, average data of patients suffering from a specific disease, or accumulated personal data of a user performing brain training.

In addition, artificial intelligence-based machine learning may be performed as follows. The brain training simulator may acquire a brain signal by measuring oxygenated, deoxygenated and total hemoglobin. In addition, the brain training simulator may remove noise components such as breathing, blood circulation, and movement of the subject from the acquired brain signal through a hemodynamic response function (hrf) and a wavelet transform algorithm. In addition, the brain training simulator may set intention data by extracting feature values by using oxygenated (Oxy-Hb), deoxygenated, and total hemoglobin values of 40 channels. The intention data may be set by repeatedly extracting feature values and performing a classification task.

The brain training simulator may determine the preset intention data matched with the brain signal data as the user intention (S1240). For example, the brain training simulator may determine whether the intention of the user is recognized, by selecting preset intention data that matches the brain signal data by a preset percentage or greater.

In addition, the brain training simulator controls the operation of the training apparatus based on the determined user intention, and controls the playback of the training content to correspond to the operation of the training apparatus (S1250). For example, the brain training simulator may control the operation of the training apparatus based on whether the intention is recognized, and control the playback of the training content displayed on the training apparatus to correspond to the operation of the training apparatus.

In detail, based on the training apparatus being a treadmill and the user intention being to walk slowly, the brain training simulator may perform driving of the treadmill slowly to correspond to user intention and the playback of the training content to also slow down so as to correspond to user intention. For example, adjusting the playback speed of the training content refers to not only adjusting the playback speed of the content itself, but also the movement speed of the avatar within the training content and the changing speed of an object within the training content.

In addition, the brain training simulator may determine, based on the selected intention data being intention data related to a second action state, that the recognition of the intention is successful, determine, based on the selected intention data being intention data related to a first action state, that the recognition of the intention is unsuccessful, based on determining that the recognition of the intention is successful, control the operation of the training apparatus for the second action state, and based on determining that the recognition of the intention is unsuccessful, control the operation of the training apparatus for the first action state.

The brain training simulator provides feedback for inducing brain activation to the user (S1260). For example, the feedback for inducing brain activation may include training state information, a message for immersion in training, an alarm indicating an improvement of a training score, or the like. The brain training simulator includes an output unit and may output feedback for inducing brain activation described above. Further, the brain training simulator may output training state feedback such as comprehensive information based on the training state information according to the operation of the training apparatus, information for dangerous situations, or the like through the output unit.

The method of controlling the brain training simulator according to various embodiments described above may be provided as a computer program product. The computer program product may include a S/W program itself or a non-transitory computer readable medium stored with the S/W program.

The non-transitory computer readable medium refers to a medium that is readable by a machine that stores data semi-permanently rather than storing data for a short time such as a register, a cache, a memory, or the like. In detail, the aforementioned various applications or programs may be stored in the non-transitory computer readable medium, for example, a compact disc (CD), a digital versatile disc (DVD), a hard disc, a Blu-ray disc, a universal serial bus (USB), a memory card, a read only memory (ROM), and the like, and may be provided.

While the disclosure has been shown and described with reference to the exemplary embodiment thereof, the present disclosure is not limited to the specific embodiments described above. It will be understood by those of ordinary skill in the art that various changes in form and details may be made therein without departing from the spirit and scope of the disclosure as defined by the appended claims and their equivalents.

What is claimed is:

1. A brain training simulator comprising:
at least one memory; and
at least one processor configured to acquire a brain signal of a user acting in a first action state based on a non-invasive brain activation measurement method, determine whether an intention of the user is recognized, by selecting preset intention data that matches data of the brain signal by a preset percentage or greater, control an operation of the training apparatus based on whether the intention is recognized, and control playback of training content displayed on the training apparatus to correspond to the operation of the training apparatus.

2. The brain training simulator according to claim 1, wherein the acquired brain signal comprises at least one of a metabolism brain signal related to exercise management of a cerebral cortex and information on an oxygen concentration of hemoglobin.

3. The brain training simulator according to claim 1, wherein the determining comprises, based on the selected intention data being intention data related to a second action state, determining that the intention is successfully recognized, and based on the selected intention data being intention data related to the first action state, determining that the intention is unsuccessfully recognized, and
the controlling of the operation of the training apparatus comprises, based on determining that the intention is successfully recognized, controlling the operation of the training apparatus for the second action state, and based on determining that the intention is unsuccessfully recognized, controlling the operation of the training apparatus for the first action state.

4. The brain training simulator according to claim 1, wherein the at least one processor is further configured to acquire training state information about an action of the user corresponding to the operation of the training apparatus, and
the training state information comprises at least one of a training distance, a training time, a number of times walking, a walking pattern, a number of times of intention recognition, a training distance based on an intention recognition, a training time based on the intention recognition, brain activation state information, biometric information about the user, a brain signal, and intention recognition information.

5. The brain training simulator according to claim 1, wherein the at least one processor is further configured to store training state information about the user in a profile corresponding to the user, and store the profile in an entire database of a patient group to which the user belongs.

6. The brain training simulator according to claim 1, wherein the at least one processor is further configured to generate an analysis data obtained by analyzing the data of the acquired brain signal of the user in real time, and generate diagnostic data on a disease of the user based on the generated analysis data and the entire database.

7. The brain training simulator according to claim 1, wherein the at least one processor is further configured to output at least one of whether the intention is recognized, training state information about an action of the user corresponding to the operation of the training apparatus, whether an operating mode of the training apparatus is changed, a message for immersion in training, and an alarm indicating an improvement of a training score.

8. The brain training simulator according to claim 1, wherein the at least one processor is further configured to output at least one of comprehensive information and information for preparing for dangerous situations, based on training state information about an action of the user corresponding to the operation of the training apparatus.

9. The brain training simulator according to claim 1, wherein the determining comprises inputting the data of the brain signal as input data to a recognition model, and determining whether the intention of the user is recognized, by acquiring, as output data, whether the intention of the user is recognized.

10. The brain training simulator according to claim 9, wherein the recognition model is trained based on an artificial intelligence-based machine learning method.

11. The brain training simulator according to claim 1, wherein the controlling of the operation of the training apparatus comprises, based on whether the intention of the user is recognized, controlling at least one of a speed, an intensity, and time of the training apparatus, a direction change within the training content, and an operation mode change of the training apparatus, while the training apparatus is in operation.

12. The brain training simulator according to claim 1, wherein the acquiring comprises, based on controlling the operation of the training apparatus for the second action state, acquiring the brain signal of the user based on the non-invasive brain activation measurement method,
the determining comprises, based on the selected intention data being intention data related to a third action state, determining that the intention is successfully recognized, and based on the selected intention data being intention data related to the second action state, determining that the intention is unsuccessfully recognized, and
the controlling of the operation of the training apparatus comprises, based on determining that the intention is successfully recognized, controlling the operation of the training apparatus for the third action state, and based on determining that the intention is unsuccessfully recognized, controlling the operation of the training apparatus for the second action state.

13. The brain training simulator according to claim 1, wherein the training content comprises at least one virtual avatar that operates based on whether the intention is recognized.

14. The brain training simulator according to claim 13, wherein the training content further comprises a virtual avatar related to an action with which the user is to be trained, and a virtual avatar related to the selected intention data.

15. A brain training simulation system comprising:
a brain training simulator configured to send training content to a training apparatus such that the training content is displayed by the training apparatus, acquire a brain signal of a user acting in a first action state based on a non-invasive brain activation measurement method, determine whether an intention of the user is recognized, by selecting preset intention data that matches data of the brain signal by a preset percentage or greater, control an operation of the training apparatus based on whether the intention is recognized, and control playback of the training content displayed by the training apparatus to correspond to the operation of the training apparatus; and
a training apparatus configured to display the training content received from the brain training simulator and operate under control of the brain training simulator.
